(19) United States
(12) Patent Application Publication
(10) Pub. No.: US 2005/0026982 A1
(43) Pub. Date: Feb. 3, 2005

(54) Composition that consists of alkanedicarboxylic acids and a pharmaceutical active ingredient

(76) Inventors: Soenke Johannsen, Berlin (DE); Thomas Zollner, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/864,757

(22) Filed: Jun. 10, 2004

Related U.S. Application Data
(60) Provisional application No. 60/477,031, filed on Jun. 10, 2003.

(30) Foreign Application Priority Data
Jun. 10, 2003 (DE) 10326722.0

(51) Int. Cl. A61K 31/4172; A61K 31/19

(52) U.S. Cl. 514/396; 514/574

(57) ABSTRACT

The invention consists of a composition that an alpha, omega-n-alkanedicarboxylic acid and an imidazole derivative with general formula 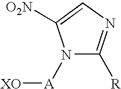 whereby R is a hydrogen atom or an alkyl group, A is a saturated chain of an aliphatic hydrocarbon group, X is a hydrogen atom or an alkyl radical of a monocarboxylic or dicarboxylic aliphatic or aromatic acid. The composition is used as a pharmaceutical active ingredient, whereby the composition is used for treating psoriasis, atopic dermatitis, common acne or rosacea.

This application claims the benefit of the filing date of U.S. Provisional Application Ser. No. 60/477,031 filed Jun. 10, 2003.

The invention relates to a composition that consists of alkanedicarboxylic acids and an additional pharmaceutical active ingredient, in particular as a pharmaceutical composition for treatment of rosacea. In addition, the invention comprises the use of the composition for the production of a medication for treatment of rosacea.

### PRIOR ART

### Composition with Azelaic Acid

German Patent Application DE 28 17 133 (application date Apr. 19, 1978) of Nazzaro-Porro describes alpha, omega-n-alkanedicarboxylic acids with 7 to 13 carbon atoms and esters thereof that can be cleaved by skin enzymes. Preferred are pimelic acid, suberic acid, azelaic acid, sebacic acid, 1,9-nonanedicarboxylic acid, 1,10-decanedicarboxylic acid and 1,11-undecanedicarboxylic acid. Azelaic acid is most preferred. The dicarboxylic acids are used for treatment of hyperpigmentary dermatoses or skin hyperpigmentations.

European Patent EP 0 305 407 (application date Jul. 7, 1987) of Nazzaro-Porro puts under protection the use of dicarboxylic acids with 7 to 13 carbon atoms, especially azelaic acid, for treatment of rosacea.

EP 0 336 880 A2, which was applied for on Mar. 29, 1998, describes a pharmaceutical composition that consists of azelaic acid at a concentration of 20% by weight and that consists of pharmaceutical additives and vehicles such as triacyl glycerides and diacyl glycerides, propylene glycol, polysorbate, for example polyethylene(20)sorbitan monooleate, and water and salts. This composition that is to be administered topically is used for treatment of various age-related changes in the facial skin. The composition exists as a cream. In addition, it is known to use the azelaic acid also against inflammatory dermatoses, such as, for example, acne and rosacea.

In the 1996 Red List (ISBN 3-87193-167-5), a pharmaceutical composition with the name Skinoren, which consists of azelaic acid at a concentration of 20% by weight and pharmaceutical vehicles and additives, such as triacyl glycerides and diacyl glycerides, propylene glycol, polysorbate, for example macrogol stearate 1000 and water and salts, is described under the number 32 282. This composition that is to be administered topically is used for treatment of acne. The composition exists as a cream.

European Patent EP 1 032 379 B1 describes a hydrogel with azelaic acid, in which the following additives are used: triacyl glycerides, propylene glycol, polyacrylic acid, soybean lecithin and polysorbates in an aqueous phase, comprising water and salts. The composition is used for the treatment of rosacea, presbyderma, melasma, acne and/or skin irritations.

### Composition with Metronidazole

U.S. Pat. No. 2,944,061 (Publication date Jul. 5, 1969) of R. M. Jacob et al. describes new imidazole derivatives that exhibit a chemotherapeutic action, in particular for treating infections with protozoa, such as pathogenic amoeba or trichomonads.

U.S. Pat. No. 4,837,378 (publication date on Jun. 6, 1989) of R. J. Borgman deals with a dermatological composition for topical administration in gel form, whereby the pharmaceutical active ingredient is metronidazole. As gel formers, polymers with free carboxyl groups, such as polyacrylic acid with a molecular weight of 1·10⁶ to 4·10⁶ Dalton, are used.

Rosacea and common acne are treated with the dermatological composition.

### Object and Achievement

The object is to offer a composition with a dicarboxylic acid as a therapeutic active ingredient, whereby an additional pharmaceutical active ingredient is also comprised. In this case, the composition of the two pharmaceutical active ingredients is to have a significantly more anti-inflammatory effect than any individual substance. In particular, the anti-inflammatory composition is to be used for treating psoriasis, atopic dermatitis, common acne and rosacea.

The object is achieved by a composition that comprises the two following substances:

(i) At least one alpha, omega-n-alkanedicarboxylic acid with 7 to 13 carbon atoms or esters thereof and

(ii) At least one imidazole derivative with general formula

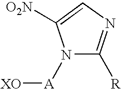

whereby

R is a hydrogen atom or an alkyl group that contains up to five carbon atoms, whereby methyl and ethyl groups are preferred,

A is a saturated, linear chain of an aliphatic hydrocarbon group that contains two to five carbon atoms,

X is a hydrogen atom or an alkyl radical of a monocarboxylic or dicarboxylic aliphatic or aromatic acid.

Preferred are alpha, omega-n-alkanedicarboxylic acids or esters thereof that are selected from the following group: pimelic acid, suberic acid, azelaic acid, sebacic acid, 1,9-nonanedicarboxylic acid, 1,10-decanedicarboxylic acid and 1,11-undecanedicarboxylic acid. More preferred is azelaic acid or esters thereof and most preferred is azelaic acid.

As ester groups, alkyl groups of 1 to 5 carbon atoms can be used independently of one another at the alpha- and omega-positions. It is essential that the esters be cleavable from the skin enzymes. Alkyl groups can be methyl, ethyl, propyl, butyl or pentyl in straight or branched form.

Preferred is an imidazole derivative from the group:

1-(2-hydroxyethyl)-2-methyl-5-nitroimidazole;

1-(2-benzoyloxyethyl)-2-methyl-5-nitroimidazole;

1-(2-succinoyloxyethyl)-2-methyl-5-nitroimidazole; and

1-(2-cinnamoyloxyethyl)-2-ethyl-5-nitroimidazole.

Most preferred is metronidazole=1-(2-hydroxyethyl)-2-methyl-5-nitroimi-dazole.

### Advantages

In the treatment of the inflammatory form of rosacea, i.a., the approved preparations metronidazole (0.5-1.0% in various vehicles) and azelaic acid (20% in various vehicles) have proven to be effective (Rebora, A. (2002) The Management of Rosacea. Am. J. Clin. Dermatol. Vol: 3(7), pp 489-96). The action of a combinatory treatment of azelaic acid and metronidazole was not previously studied in vivo; only data in the comparison of the activity of individual substances in the case of rosacea exist (Maddin (1999) Journal of the American Academy of Dermatology, Volume 40(69, pp. 961- 965). In addition, no study results that make an additive or synergistic action of a combination therapy likely have been published.

It was found, surprisingly enough, that a combination therapy of metronidazole (0.75%) and azelaic acid (20%) in ethanol/isopropyl myristat in an inflammation model is significantly therapeutically effective in comparison to the vehicle, while the two individual substances metronidazole (0.75%) or azelaic acid (20%) do not show such high, therapeutic effects in comparison to the vehicle. The activity included, i.a., the immigration of leukocytes, measured as peroxidase activity in the skin, as well as the immigration of neutrophilic granulocytes, measured as elastase activity in the skin. The immigration of this cell population in the skin and the release of pro-inflammatory substances is involved in the pathogenesis of a number of inflammatory diseases. These diseases contain, i.a., rosacea, acne, allergic and irritative contact dermatitis, atopic dermatitis and psoriasis (Braun-Falco, O., G. Plewig et al. (1995) Dermatologie und Venerologie [Dermatology and Venereology], Heidelberg, Springer).

The induction of an inflammatory reaction (irritative contact dermatitis) is a common model for pharmacological evaluation of the anti-inflammatory potential of topically or systemically administered test substances (Trancik and Lowe (1985) Evaluation of Topical Nonsteroidal Anti-Inflammatory Agents, Models in Dermatology, H. Maibach and N. J. Lowe, Basel Karger: 35-42). It has proven effective, i.a., in the testing of non-steroidal antiphlogistic agents as well as in glucocorticoid steroids and is regarded as the most reproducible in-vivo experiment for the evaluation of topical anti-inflammatory substances (Lorenzetti (1975) Animal to Clinical Correlation of Topical Anti-Inflammatory Efficacy, Animal Models in Dermatology. H. Maibach, Basel Karger: 212-224).

### Additional Embodiments

More preferred is a composition according to the invention that comprises a pharmaceutical composition with pharmaceutical vehicles and adjuvants that are described in Remington's Pharmaceutical Science, 15^{th} Ed. Mack Publishing Company, Easton, Pa. (1980).

### Additional Object and Achievement

In addition, the bioavailability of the two active ingredients is to be increased.

The pharmaceutical composition consists of at least:

One alpha, omega-n-alkanedicarboxylic acid with 7 to 13 carbon atoms or esters thereof, and

at least one imidazole derivative with the previously mentioned general formula

with the following pharmaceutical vehicles:

Triacyl glycerides, propylene glycol, polysorbates, polyacrylic acid, soybean lecithin in an aqueous phase, comprising water and salts, whereby the composition is a hydrogel.

### Advantages

The composition according to the invention exhibits the advantage that it allows a larger amount of pharmaceutical active ingredient to penetrate into living skin layers and/or cutaneous organs. This availability can be detected in a FRANZ-flow-diffusion cell test.

### Another Embodiment of the Composition

A composition that can be administered topically is advantageous.

The presence of polyacrylic acid is essential. It is decisive for the production of the hydrogel. In the gel, soybean lecithin is preferred as lecithin. The lecithin or soybean lecithin is advantageous at a concentration of at least 3% by weight. More preferred is a concentration of at least 1.5% by weight, and most preferred is a concentration of at least 1% by weight.

### Advantages

The composition according to the invention advantageously has a high penetration into living skin layers and/or cutaneous organs.

### Preferred Embodiments

Preferred is a composition according to the invention in which the individual parameters, independently of one another, can have the following concentrations:

Polyacrylic acid at a concentration of 0.5 to 2% by weight,

Triacyl glycerides at a concentration of 0.5 to 5% by weight,

Propylene glycol at a concentration of 5 to 15% by weight, and

Polysorbates at a concentration of 0.5 to 3% by weight.

The components are to be coordinated with one another, of course, so that a sum of 100% is achieved.

Most preferred is a composition in which the individual parameters, independently of one another, can exhibit the following concentrations:

Polyacrylic acid at a concentration of 1±0.25% by weight,

Triacyl glycerides at a concentration of 2±1% by weight,

Propylene glycol at a concentration of 10±2% by weight, and

Polysorbates at a concentration of 2±0.5% by weight.

The components are to be coordinated with one another, of course, so that a sum of 100% is achieved.

### Definitions

Azelaic acid and production thereof is described in German Patent DE 28 17 133.7. Cf. also Römpp, Chemie Lexikon [Lexicon of Chemistry], published by Jürgen FALBE and Manfred REGNITZ, 1989, 9^{th} Edition, page 323, Thieme Verlag Stuttgart, ISBN 3-13-734609-6.

Metronidazole and the production thereof is described in the U.S. Pat. No. 2,944,061 (publication date Jul. 5, 1969) of R. M. Jacob et al. New imidazole derivatives and, in particular, metronidazole, are characterized therein. U.S. Pat. No. 4,837,378 (publication date on Jun. 6, 1989) of R. J. Borgman deals with a topical administration of metronidazole. Rosacea and common acne are treated with the dermatological composition.

Polyacrylic acid represents an anion-active polymerizate that consists of acrylic acid, which is only partially soluble in water. The 1% aqueous suspension has a pH of 3 and approximately the same viscosity as water. Only during neutralization with inorganic or organic bases do gel formation and a production of highly viscous products result. Rudolf VOIGT and Manfred BORNSCHEIN, 1979, Lehrbuch der pharmazeutischen Technologie [Textbook of Pharmaceutical Technology], page 314, VEB Verlag Volk und Gesundheit Berlin. Cf. also Römpp, Chemie Lexikon, published by Jürgen FALBE and Manfred REGNITZ, 1992, 9^{th} Edition, page 3508, Thieme Verlag Stuttgart, ISBN 3-13-735009-3.

“Triglyceride” is a designation for glycerol ester, whose three hydroxy groups are esterified by carboxylic acids. The naturally occurring fats and fatty oils are triglycerides (“neutral fats”), which generally contain various fatty acids in the same glycerol molecule. J. Am.- Oil. Chem Soc. Volume 62, page 730, (1985); and Parfüm. Kosmet. Volume 58, page 353, (1977); and Römpp, Chemie Lexikon, published by Jürgen FALBE and Manfred REGNITZ, 1990, 9^{th} Edition, pages 1339-1342, Thieme Verlag Stuttgart, ISBN 3-13-734709-2.

Propylene glycol is described in H. P. FIEDLER: Lexikon der Hilfsstoffe [Lexicon of Adjuvants], 4^{th} Edition, 1996, ISBN 3-87193-173 on pages 1278 to 1282.

Polysorbates are described in H. P. FIEDLER: Lexikon der Hilfsstoffe, 4^{th} Edition, 1996, ISBN 3-87193-173 on pages 1251 to 1252.

Lecithins are obtained by extraction from biological material. A lecithin fraction that consists of soybeans (the most common raw material) thus comprises, e.g., palmitic acid, stearic acid, palmitoleic acid, oleic acid, linoleic acid or linolenic acid. Normally, the saturated fatty acid is esterified with the primary hydroxy group of the glycerol, and the unsaturated fatty acid is esterified with the secondary hydroxy group of the glycerol. Lecithins are components of the cell membranes of all living creatures. In water, lecithins first swell like lyophilic colloids. Later, they produce transparent, colloidal solutions. Depending on the water content, they form different textures, whereby the lamellae are formed from lipid double layers. At a higher water content, liposomes are produced. Lit.: Pardun, Die Pflanzenlecithine [The Plant Lecithins], Augsburg: Verl. füir Chem. Ind. (Ziolkowsky KG) 1988. Additional lecithins and action thereof are described in J. GAREISS et al. 1994, Parfümerie und Kosmetik [Perfumes and Cosmetics], Vol. October 1994, pages 652-659, Hüthing GmbH Heidelberg.

A gel is distinguished by the following properties: It is a dimensionally stable, easily deformable, liquid and optionally gas-rich, dispersed system that consists of at least two components. Römpp, Chemie Lexikon, published by Jürgen FALBE and Manfred REGNITZ, 1990, 9^{th} Edition, page 1511, Thieme Verlag Stuttgart, ISBN 3-13-734709-2; and Pharm. Unserer Zeit [Pharmaceutics of Our Time], Vol. 8, pages 179 to 188, (1979); and Parfüm. Kosmet., Vol. 58, pages 251 to 253 (1977).

Preservatives can be contained in the aqueous phase. The preservatives include, for example, benzoic acid. Based on the antimicrobial property thereof, benzoic acid is especially suitable as a preservative.

### Properties When Used as a Medication

### Pharmaceutical Active Ingredient

The composition of the invention shows pharmacological action in an established testing system for evaluating topical anti-inflammatory substances (0. J. Lorenzetti (1975) Animal to Clinical Correlation of Topical Anti-Inflammatory Efficacy. Animal Models in Dermatology, H. Maibach, Basel, Karger, pp. 212-225). The composition can therefore be used as a therapeutic active ingredient or as a medication.

The composition of the invention is suitable for treating various indications. Preferred is a composition of the invention as a therapeutic active ingredient for treating psoriasis, atopic dermatitis, common acne and/or rosacea. In addition, the invention comprises the use of a composition according to the invention for the production of a medication for treating psoriasis, atopic dermatitis, common acne and/or rosacea.

More preferred is a composition of the invention as a therapeutic active ingredient for treating rosacea or psoriasis together with at least one physiologically compatible, pharmacological adjuvant or vehicle.

(i) In addition, the invention provides

(α) the use of the pharmaceutical composition of the invention for the production of a medication for treating psoriasis, atopic dermatitis, common acne and/or rosacea;

(β) a process for treating psoriasis, atopic dermatitis, common acne and/or rosacea, said process comprises an administration of a pharmaceutical composition according to the invention, whereby the amount suppresses the disease, and whereby the pharmaceutical composition is given to a patient who requires such a medication;

(γ) a pharmaceutical composition for treating psoriasis, atopic dermatitis, common acne and/or rosacea, said treatment comprises a pharmaceutical composition of the invention and at least one pharmaceutically compatible vehicle and additive.

For these therapeutic actions, the suitable dose is different and depends on, for example, the concentration of the individual elements in the pharmaceutical composition, the host, the type of administration and the type and severity of the conditions to be treated.

Preferred concentrations of alpha, omega-n-alkanedicarboxylic acids with 7 to 13 carbon atoms, especially azelaic acid, lie in the range of 2 to 40% (% by weight), more preferably 5 to 20%, still more preferably 8 to 17%, and most preferably 10-15%. Preferred is a concentration of imidazole derivative and esters thereof, in particular metronidazole, of at least 0.1% to 3% (% by weight), more preferably 0.25% to 1%, and most preferably 0.7% to 0.8%.

The treatment comprises prophylactic and therapeutic measures. The components of the composition can be administered in a dosage at the same time and/or space; in addition, they can be administered separated from one another physically and/or in time.

### Pharmaceutical Preparations

The pharmaceutical composition of the invention can be processed into the commonly used topical forms of administration with the additives and/or vehicles that are commonly used in galenical pharmaceutics according to methods that are known in the art. Preferred is a composition according to the invention together with at least one physiologically compatible, pharmacological adjuvant or vehicle. Pharmacological adjuvants and vehicles are described in Remington's Pharmaceutical Science, 15^{th} Ed. Mack Publishing Company, Easton, Pa. (1980).

In the case of topical treatment, the pharmaceutical composition of the invention can be administered in any commonly used method by the active ingredients being converted with suitable additives into the desired form of administration, such as, for example, solutions, lotions, creams, ointments, pastes or gels. The gel is preferred. The lotions, creams, ointments and gels can be produced in a conventional way with use of conventional emulsifiers. (Kirk Othmer: Encyclopedia of Chemical Technology, 3^{rd} Edition, 1979, John Wiley & Sons, New York, Vol. 8, pages 900-930, and Dr. Otto-Albrecht Neumüller: Römpps Chemie Lexikon, 7^{th} Edition, 1973 Franckh' sche Verlagshandlung [Franckh Publishing House], Stuttgart, pages 1009-1013).

Keratolytic agents, such as, for example, salicylic acid, vitamin A acid, resorcinol, phenol, cresol and the like, can be added to the pharmaceutical agent.

The physiologically compatible salts include alkali metal salts, such as sodium and potassium salts, also salts with basic amino compounds and organic amines, such as, for example, arginine, lysine or N-methyl glutamine.

As hydrophilic and/or lipophilic additives, moisturizing factors (hydro complexes), such as, for example, propylene glycol, glycerol, polyethylene glycols or amino acid mixtures, Puroba oil (=jojoba oil), vitamins (preferably vitamins A and E), vital complexes (such as, for example, placenta extracts), enzymes, herb extracts (such as, for example, hamamelis extract or chamomile extract) or proteins (such as, for example, collagen) can be used. Hydrocarbons, such as, for example, squalene, vaseline, paraffins or stearin, or waxes, such as, for example, beeswax, are suitable as an oily phase or as a fatty phase. Suitable oil/water emulsifiers are, for example, stearyl alcohol, polyoxyethylene stearates (such as, for example, MYRJ®), complex emulsifiers (such as, for example, Amphoterin®) and sorbitan fatty acid esters (such as, for example, Tween 80®) or carboxyvinyl polymerizates (such as, for example, Carbopol®). In addition, the aqueous phase can also contain buffer substances, such as, for example, the disodium salt of ethylenediamine-N,N,N′,N′-tetraacetic acid, and preservatives, such as benzoic acid, chloroquinaldol, parabene or benzalkonium chloride.

### Indications

### Rosacea

Rosacea is an initially chronic hyperemic (Rosacea I), later chronically inflammatory (Rosacea II and III) disease of the face. It can be attributed to elevated vascular reactions as well as inflammatory processes with immigration of leukocytes and release of inflammatory mediators. It is distinguished by a persistent erythema with acute inflammation phases with the formation of edemas, papules and pustules. It usually occurs at over 20 years of age. Azelaic acid, antibiotics and corticosteroids are used for treatment.

The successful treatment of rosacea is difficult. The composition according to the invention is suitable to exert an advantageous effect both on the inflammatory components (papules and pustules) as well as on the vascular components of rosacea (erythema and telangiectasia).

### Acne

The composition according to the invention shows good action in the treatment of acne. Acne is an abnormality of the oil glands with inflammatory papules, pustules and cysts and with non-inflammatory blackheads. In most cases, it affects adolescents and young adults.

The composition according to the invention is suitable to exert an advantageous effect on acne. The advantageous therapeutic effect of the composition according to the invention is shown in a clear reduction of papules, pustules and cysts.

### Psoriasis

Psoriasis is a common, chronic, proliferative skin disease in which the keratinocytic epidermal passage time is considerably increased. The lesions are sharply delimited, thick, erythematous spots with proliferating white scaling. The elbows, the knees, the scalp, the genitals and the gluteal folds are usually affected.

The composition according to the invention is suitable to exert an advantageous effect on the lesions.

### EXAMPLE

The induction of an inflammatory reaction (irritative contact dermatitis) is a common model for pharmacological evaluation of the anti-inflammatory potential of topically or systemically administered test substances (Trancik and Lowe (1985) Evaluation of Topical Nonsteroidal Anti-Inflammatory Agents, Models in Dermatology, H. Maibach and N. J. Lowe. Basel, Karger: 35-42). It has proven effective, i.a., in the testing of non-steroidal antiphlogistic agents as well as in glucocorticoid steroids and is regarded as the most reproducible in-vivo test for the evaluation of topical anti-inflammatory substances (Lorenzetti (1975) Animal to Clinical Correlation of Topical Anti-Inflammatory Efficacy. Animal Models in Dermatology. H. Maibach. Basel Karger: 212-225).

The application of the phorbol ester croton oil to mouse ears results in an acute, inflammatory reaction with edema and immigration of primarily polymorphonuclear granulocytes that reaches its maximum within 24 hours. In this connection, test substances can be administered either preventively (i.e., before administration of the inflammatory stimuli) or simultaneously with croton oil. The croton oil-induced inflammation on the mouse ear is not only a model of irritative and/or allergic contact dermatitis, but also other skin diseases that are associated with a high inflammation and immigration of granulocytes. These include psoriasis, atopic dermatis, common acne and the inflammatory stage of rosacea.

Rosacea is a disease of unknown etiology that is considered by many authors to be in the acne group and is generated on the latter and can remove the latter over the course of time. The disease is divided into three stages, of which in particular Rosacea II and III are characterized by inflammatory nodules, nodes, and patches. A leukocytic infiltrate is found by histology (Braun-Falco et al. (1995) Dermatologie und Venerologie. Heidelberg, Springer). Because of the inflammatory components of these stages with leukocyte infiltration of the skin, the croton oil model reflects aspects of this disease.

In the treatment of the inflammatory form of rosacea, i.a., the approved preparations metronidazole (0.5-1.0% in various vehicles) and azelaic acid (20% in various vehicles) have proven to be effective (Rebora (2002) The Management of Rosacea. Am. J. Clin. Dermatol. Vol: 3(7), pp. 489-498). The action of a combinatory treatment of azelaic acid and metronidazole was not previously studied in vivo; only data in the comparison of the activity of individual substances in the case of rosacea exist (Maddin (1999) A Comparison of Topical Azelaic Acid 20% Cream and Topical Metronidazole 0.75% Cream in the Treatment of Patients with Papulopustular Rosacea. Journal of the American Academy of Dermatology, Volume 40(6): pp. 961-965). In addition, no study results that lead one to expect an additive or synergistic action of a combination therapy have been published.

It was found, surprisingly enough, that a combination therapy of metronidazole (0.75%) and azelaic acid (20%) in ethanol/isopropyl myristat was significantly therapeutically effective in comparison to the vehicle, while the two individual substances metronidazole (0.75%) or azelaic acid (20%) did not show such high, therapeutic effects in comparison to the vehicle. The activity included, i.a., the immigrations of leukocytes, measured as peroxidase activity in the skin, as well as the immigration of neutrophilic granulocytes, measured as elastase activity in the skin (Table 1).

The immigration of this cell population in the skin and the release of pro-inflammatory substances is involved in the pathogenesis of a number of inflammatory diseases. These diseases contain, i.a., rosacea, acne, allergic and irritative contact dermatitis, atopic dermatitis and psoriasis. Braun-Falco et al. (1995) Dermatologie und Venerologie, Heidelberg, Springer.

The leukocyte infiltration into the croton-oil-inflamed mouse skin is significantly inhibited by the combined treatment with azelaic acid and metronidazole in comparison to the vehicle treatment, while the individual active ingredients were not so effective.

TABLE 1

| | | Inhibition [%] of the Skin Infiltration * of | |
|---|---|---|---|
| Dissolving- | | Neutrophilic | Granulocytes & Monocytes |
| out | Treatment | Elastase Activity | (Myelo-Peroxidase Activity) |
| Vehicle | BVehicle | 100 | 100 |
| Croton Oil | Vehicle | 0 | 0 |
| | (Negative Control) | | |
| Croton Oil | Azelaic Acid (20%) | 11 (n.s.)* | 21 (n.s.)* |
| | (Individual | | |
| | Substance) | | |
| Croton Oil | Metroidazole | 18 (n.s.)* | 9 (n.s.)* |
| | (0.75%) | | |
| | (Individual | | |
| | Substance) | | |
| Croton Oil | Azelaic Acid (20%) + | 68 (p < 0.05)* | 46 (p < 0.05)* |
| | Metronidazole | | |
| | (0.75%) | | |
| | (Composition | | |
| | According to the | | |
| | Invention) | | |
| Croton Oil | Methylprednisolone | 95 (p < 0.05)* | 90 (p < 0.05)* |
| | (0.1%) | | |
| | (Positive Control) | | |

*Inhibition of the leukocyte infiltration in comparison to the croton oil-dissolving-out and vehicle treatment (i.e., in comparison to the untreated positive control, whereby a 100% inhibition corresponds to the value of the vehicle dissolving-out and vehicle therapy, i.e., untreated negative control).

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding German application No. 10326722.0, filed Jun. 10, 2003, and U.S. Provisional Application Ser. No. 60/477,031, filed Jun. 10, 2003, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Composition that comprises the following two substances:
(i) At least one alpha, omega-n-alkanedicarboxylic acid with 7 to 13 carbon atoms or esters thereof and
(ii) at least one imidazole derivative with general formula 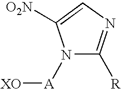
whereby
R is a hydrogen atom or an alkyl group that contains up to five carbon atoms,
A is a saturated, linear chain of an aliphatic hydrocarbon group that contains two to five carbon atoms,
X is a hydrogen atom or an alkyl radical of a monocarboxylic or dicarboxylic aliphatic or aromatic acid.

2. Composition according to claim 1, whereby the alpha, omega-n-alkane-dicarboxylic acid is azelaic acid.

3. Composition according to claim 1, whereby the imidazole derivative is a 1-(2-hydroxyethyl)-2-methyl-5-nitroimidazole.

4. Composition according to claim 1, whereby the alpha, omega-n-alkanedicarboxylic acid or esters thereof and the imidazole derivative comprise the following pharmaceutical vehicles:
triacyl glycerides, propylene glycol, polysorbates, polyacrylic acid, soybean lecithin in an aqueous phase, comprising water and salts.

5. Composition according to claim 4, whereby the individual vehicles, independently of one another, exhibit the following concentrations:
Polyacrylic acid at a concentration of 0.5 to 2% by weight,
Triacyl glycerides at a concentration of 0.5 to 5% by weight,
Propylene glycol at a concentration of 5 to 15% by weight, and
Polysorbates at a concentration of 0.5 to 3% by weight.

6. Composition according to claim 1 as a pharmaceutical active ingredient.

7. Composition according to claim 6 for treating psoriasis, atopic dermatitis, common acne and/or rosacea.

8. Composition according to claim 6, whereby the concentration of alpha, omega-n-alkanedicarboxylic acid lies in the range of 2 to 40% (% by weight), and the concentration of imidazole derivative lies in the range of at least 0.1% to 3% (% by weight).

9. Composition according to claim 1 with pharmaceutical additives and/or vehicles.
